(19) United States
(12) Patent Application Publication
(10) Pub. No.: US 2026/0174689 A1
(43) Pub. Date: Jun. 25, 2026

(54) READY-TO-USE MORPHINE COMPOSITION AND METHODS

(71) Applicant: Nivagen Pharmaceuticals, Inc., Sacramento, CA (US)

(72) Inventors: Niravbhai Jayantibhai Patel, Sacramento, CA (US); Dasaradhi Dnyanu Lakkaraju, Sacramento, CA (US); Robert Miller, Sacramento, CA (US); Govind Rangnath Jagadale, Sacramento, CA (US); Jay Shukla, Sacramento, CA (US)

(73) Assignee: Nivagen Pharmaceuticals, Inc., Sacramento, CA (US)

(21) Appl. No.: 18/990,649

(22) Filed: Dec. 20, 2024

(51) Int. Cl. A61K 9/08; A61K 9/00; A61K 31/485; A61K 47/02; A61K 47/12; A61K 47/18

(52) U.S. Cl. CPC A61K 9/08; A61K 9/0019; A61K 31/485; A61K 47/02; A61K 47/12; A61K 47/183

(57) ABSTRACT

A ready-to-administer parenteral liquid formulations comprising morphine or a pharmaceutically acceptable salt thereof, methods for preparing such formulations, methods for using such formulations, and polymeric infusion container filled with such formulations.

### TECHNICAL FIELD

The present invention relates to a ready-to-use, or ready-to-administer, parenteral liquid formulation comprising morphine or a pharmaceutically acceptable salt thereof, a method for preparing such formulation, a method for using such formulation. The ready-to-administer morphine formulation can be provided in a polymeric infusion container.

### BACKGROUND

Morphine, formerly also called morphia, is an opiate that is found naturally in opium, a dark brown resin produced by drying the latex of opium poppies (Papaver somniferum); it is classified pharmacologically as a narcotic analgesic. Morphine is indicated for the management of pain severe enough to require an opioid analgesic and for which alternate treatments are inadequate. Morphine is commonly used in the sulfate salt form.

Current morphine sulfate products include an aseptically filled liquid product packaged in prefilled syringes and vials. No ready-to-use continuous intravenous infusion is available in the market. Current marketed products are clearly mentioned on this label “NOT FOR DIRECT INJECTION” and “FOR INTRAVENOUS USE ONLY AFTER DILUTION”. An example of such labeling is shown in FIG. 1.

Opioids such as morphine are not commercially available in volumes larger than what is currently provided by the approved formulations. To meet the need of patients that require longer term pain management and/or lower concentrations than commercially available, hospital and health system pharmacies have to prepare larger volumes of morphine.

Compounding larger volumes of morphine to fill this gap of availability raises several concerns. For example, dilution of commercially available morphine sulfate products can introduce error in mixing and measuring diluents or other components. Dilution of commercially available products increases the risk for contamination and may compromise sterility. Dilution of commercially available products also suffers from the disadvantage of time-consuming dilution steps that introduce potential undesirable impurities with the use of external components.

Recommended storage and handling for marketed morphine products include “Store at 20° C. to 25° C. (68° F. to 77° F.) [See USP Controlled Room Temperature] until ready to use. PROTECT FROM LIGHT. DO NOT FREEZE. Contains no preservative or antioxidant. DISCARD ANY UNUSED PORTION. All marketed products clearly recommended “DO NOT HEAT-STERILIZE.”.”

Thus, there remains a need for ready-to-administer injectable formulations of morphine, or salts thereof, that offer long-term storage stability and can be terminally sterilized after filling into its storage container.

### SUMMARY OF THE INVENTION

The subject invention is directed to ready-to-administer parenteral liquid formulations comprising morphine or a pharmaceutically acceptable salt thereof such as morphine sulfate. These ready-to-administer formulations can advantageously be terminally sterilized in a polymeric infusion container and are stable upon long-term storage. As disclosed herein, terminally sterilized morphine sulfate liquid formulations in polymeric infusion containers are stable upon long-term storage at room temperature such as from about 20° C. to about 25° C.

In one embodiment, the invention is a ready-to-administer parenteral liquid formulation comprising:
(a) morphine sulfate;
(b) a chelating agent;
(c) a tonicity adjusting agent; and
(d) optionally, a buffer system.

Preferably, this liquid formulation has a pH of 2.5 to 4.0. The liquid can be contained and stored in a polymeric infusion container, and terminally sterilized. Advantageously, this described liquid formulation is stable for at least 6 months when stored at 25° C.±2° C. and 40% relative humidity (RH)±5% RH. A stable liquid formulation according to the invention can retain at least 95% w/w of the morphine sulfate and has not more than 5% w/w of total related substances after storage at 25° C.±2° C. and not more than 40% RH for at least 6 months. More preferably, a stable liquid formulation according to the invention can retain at least 98% w/w of the morphine sulfate and has not more than 2% w/w of total related substances after storage at 25° C.±2° C. and not more than 40% RH for at least 6 months.

The optional buffer system in the described formulation can comprise, if present, a citrate buffer, wherein the citrate buffer can comprise about 0.02% (w/v) of disodium edetate and about 0.04% (w/v) of citric acid monohydrate.

It is preferred that the liquid formulation of the invention has an osmolality of from about 275 mOsmol/kg to about 310 mOsmol/kg. Tonicity can be adjusted using a tonicity agent, such as sodium chloride, and more preferably about 9 mg/ml sodium chloride.

In one preferred embodiment, the liquid formulation of the invention comprises from about 0.05 mg/ml to about 1.5 mg/ml of morphine sulfate. In one embodiment, a stable liquid formulation according to the invention comprises about 1.0 mg/ml of morphine sulfate.

In another embodiment, the strength, or concentration of active ingredient, for a liquid formulation of the invention can be substantially less than 1.0 mg/ml, and can comprise from about 0.05 mg/ml to about 0.5 mg/ml of morphine sulfate. This low-strength embodiment can comprise about 0.2 mg/mL. A low-strength liquid formulation of the invention preferably has a pH from about 3.0 to about 3.3, which can be attained using a pH adjusting agent, such as an acid, e.g., HCl, or base, e.g., NaOH.

A method of the invention comprises providing a ready-to use liquid formulation comprising morphine, and parenterally administering the liquid morphine formulation to a patient in need thereof for treating pain. In a preferred embodiment, the formulation is administered to a patient suffering from moderate to severe pain, e.g., pain due to surgery, cancer, trauma or burns.

In one preferred embodiment, the invention comprises an administration kit comprising a liquid morphine formulation described herein, contained within a polymeric infusion container. The liquid formulation of the invention can be terminally sterilized within the polymeric infusion container, and can provide stability for the liquid formulation for about 6 months or more.

In one aspect, the invention comprises a terminally sterilized solution comprising morphine sulfate in 0.9% sodium chloride packaged in a ready-to-administer (RTA) intravenous (IV) bag. In certain preferred embodiments, the concentration of morphine sulfate is 1 mg/ml, such as 50 mg in 50 mL fill or 100 mg in 100 mL packaged in a 100 mL RTU IV bag.

The ready-to-administer parenteral liquid formulation of the invention can comprise morphine sulfate; a tonicity adjusting agent; an optional (with or without) buffer system; an optional chelating agent. One preferred embodiment of a liquid formulation of the invention comprising about 1 mg/ml morphine sulfate has a pH of 2.5 to 4.0 The liquid formulation of the invention is preferably terminally sterilized in a polymeric infusion container, and is stable for at least 6 months when stored at 25° C.±2° C. and not more than 40% RH.

The invention further includes a method for making the ready-to-administer parenteral liquid formulations described herein. The method comprises preparing a bulk solution comprising morphine sulfate, filling the bulk solution into a polymeric infusion container, and terminally sterilizing the bulk solution in the polymeric infusion container.

The invention also includes a method for the management of pain using the ready-to-administer parenteral liquid formulations described herein. The methods comprise parenterally administering by infusion to a subject in need of management of pain the ready-to-administer parenteral liquid formulations described herein.

The invention also concerns an administration kit for infusion of a morphine composition to a patient in need thereof. An administration kit according to the invention comprises a polymeric infusion container filled with the ready-to-administer parenteral liquid formulations described herein. The polymeric infusion containers and their contents can be terminally sterilized, without harm to the morphine sulfate composition contained therein and without negatively affecting the stability of the morphine sulfate composition.

These and other objects of the invention are described in detail in the following paragraphs. These objects should not be deemed to narrow the scope of the invention.

### BRIEF DESCRIPTION OF THE DRAWINGS

FIG. 1 shows a label of an approved morphine sulfate product, illustrating the express instruction “FOR INTRAVENOUS USE ONLY AFTER DILUTION.”

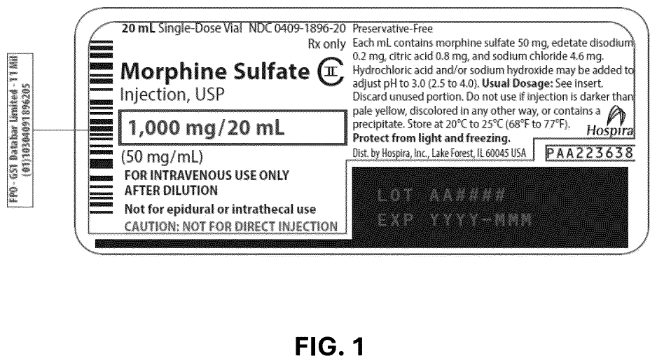

FIG. 2 is a process flow diagram for the manufacture of a terminally sterilized morphine sulfate liquid formulation in a ready-to-administer bag.

FIG. 3 shows the chemical formula of morphine sulfate (pentahydrate).

### DETAILED DESCRIPTION OF THE INVENTION

This detailed description is intended to describe the present invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent application, and may be variously modified.

### A. Definitions

As used in this specification, including the drawings, and the appended claims, unless specified to the contrary, the following terms have the meaning indicated:

The term “about” refers generally to a range of numerical values (e.g., +5 to 10% of the recited value) that one of ordinary skill in the art would consider equivalent to the recited value. Ranges can be expressed herein as from “about” one particular value, and/or to “about” another particular value. When such a range is expressed, the range is inclusive of the recited values.

The term “initial” when used in connection with a given parameter such as osmolality, pH, morphine sulfate content, or impurities refers to that parameter at the time the morphine sulfate composition is filled into the polymeric infusion container, prior to subjecting the polymeric infusion container and morphine sulfate composition to terminal sterilization.

The term “pharmaceutically acceptable” is used adjectivally to mean that the modified noun is appropriate for use as a pharmaceutical product for human use or as a part of a pharmaceutical product for human use.

The term “subject” includes humans and other primates as well as other mammals. The term subject includes, for example, a patient, such as a cancer patient or a post-operative patient, suffering from or believed to be suffering from moderate to severe pain. In certain embodiments, the subject is a human.

The terms “treat”, “treating” and “treatment” refer to a method of alleviating, abrogating, managing, or mediating a condition, disorder, or disease and/or the attendant symptoms thereof.

### B. Drug Substance

Liquid formulations disclosed herein comprise at least one active pharmaceutical ingredient: morphine or a pharmaceutically acceptable salt thereof. In one particular embodiment, the pharmaceutically acceptable salt of morphine is morphine sulfate.

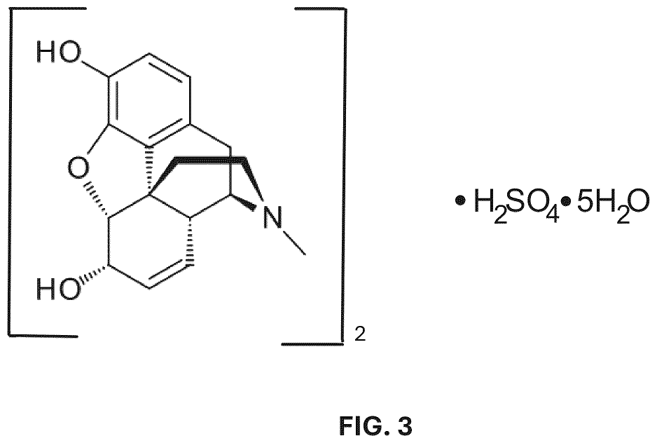

Morphine sulfate is chemically identified as 7,8-didehydro-4,5α-epoxy-17-methylmorphinan-3,6α-diol sulfate (2:1) (salt), pentahydrate. The molecular weight of morphine sulfate pentahydrate is 758.83 g/mol and its molecular formula is (C₁₇H₁₉NO₃)₂·H₂SO₄·5H₂O. The chemical structure of morphine sulfate is shown in FIG. 3.

Other suitable morphine salts include any water-soluble salt of morphine such as morphine sulfate.

In certain embodiments, morphine sulfate is present in the liquid formulation in an amount from about 0.02 mg/mL to about 1.5 mg/mL. In certain embodiments, the liquid formulation comprises 1.0 mg/ml of morphine sulfate, USP. In certain embodiments, the liquid formulation comprises about 0.2 mg/mL.

### C. Liquid Formulations

The subject invention comprises a liquid formulation that is suitable for terminal sterilization in a polymeric infusion container and is stable upon long-term storage following such terminal sterilization. The liquid formulation may contain one or more excipients such as a tonicity adjusting agent, a chelating agent, a pH adjusting agent, a buffer system and/or a preservative. The chelating agent, pH adjusting agent, buffer system and preservative are optional components and may or may not be present according to need.

In certain embodiments, the disclosed liquid formulation comprises a tonicity adjusting agent and/or a chelating agent.

In certain embodiments, the liquid formulation is isotonic or slightly hypotonic. In some such embodiments, the liquid formulation is isotonic.

In certain embodiments, the liquid formulation has an osmolality in the range from about 250 mOsmol/kg to about 375 mOsmol/kg, alternatively, about from about 270 mOsmol/kg to about 330 mOsmol/kg, or, alternatively, from about 285 mOsmol/kg to about 310 mOsmol/kg. In an exemplary embodiment, the osmolality of the liquid formulation is about 289 mOsmol/kg.

In certain embodiments, the osmolality of the liquid formulation is adjusted by addition of a tonicity adjusting agent. The tonicity adjusting agent that may be, without limitation, sodium chloride, calcium chloride, mannitol, glycerol, sorbitol, propylene glycol, dextrose, sucrose, and the like and mixtures thereof. In certain embodiments, the tonicity adjusting agent comprises sodium chloride, dextrose, and sucrose. In some such embodiments, the tonicity adjusting agent is sodium chloride.

In certain embodiments, the tonicity adjusting agent is sodium chloride. In some such embodiments, the liquid formulation comprises from about 0.5% w/v to about 1.2% w/v sodium chloride. In an exemplary embodiment, the liquid formulation comprises about 0.9% w/v sodium chloride.

In certain embodiments, the liquid formulation has an initial pH in the range of about 2.0 to about 6.5, 2.5-6.5, alternatively from about 2.5 to about 4.5, 2.5-4.5. In an exemplary embodiment, the liquid formulation has an initial pH in the range of 2.5 to about 4.0, preferably about 3.0

In certain embodiments, the disclosed liquid formulation comprises a pH adjusting agent. In certain embodiments, the pH of the liquid formulation is adjusted by the addition of a pH adjusting agent. The pH adjusting agent that may be, without limitation, an acid such as sulfuric acid, phosphoric acid, or hydrochloric acid, a base such as sodium acetate, sodium bicarbonate, or sodium hydroxide, or a combination thereof.

In certain embodiments, the pH adjusting agent is hydrochloric acid, sodium hydroxide, or a combination thereof. The concentration of the pH adjusting agent can be any concentration suitable for adjusting the pH, such as, for example, 0.1 N acid or base. In certain embodiments, 0.1 N hydrochloric acid and/or 0.1 N sodium hydroxide are added to obtain the desired pH.

In certain embodiments, the disclosed liquid formulation comprises a buffer system. In certain embodiments, the pH of the liquid formulation is maintained within the preferred range by addition of a buffer system. The buffer system may be, without limitation, a phosphate buffer, a citrate buffer, an acetate buffer, a histidine buffer, or a combination thereof. In certain embodiments, the buffer system comprises a citrate buffer. In some such embodiments, the liquid formulation comprises from about 0.1 mg/ml to about 1.0 mg/ml of the citrate buffer.

In certain embodiments, the buffer system comprises sodium citrate, citric acid, or a combination thereof. Examples of sodium citrate and citric acid include anhydrous forms as well as hydrates, such as sodium citrate dihydrate, trisodium citrate anhydrous, trisodium citrate dihydrate, trisodium citrate pentahydrate, citric acid anhydrous, and citric acid monohydrate.

In certain embodiments, the buffer system comprises sodium citrate dihydrate and citric acid monohydrate. In some such embodiments, the liquid formulation comprises from about 0.01% w/v to about 0.1% w/v sodium citrate dihydrate and from about 0.01% w/v to about 0.1% w/v citric acid monohydrate. In an exemplary embodiment, the liquid formulation comprises 0.04% (w/v) of citric acid monohydrate.

In certain embodiments, the buffer system stabilizes the pH of the liquid formulation. Thus, for example, a pH adjusting agent and/or a buffer system may be present in the liquid formulation in an amount sufficient to provide and maintain a 2.5 to about 6.5 pH of about 2.5 to about 4.0, alternatively, from about 2.5-4.0, or alternatively from about 3.0.

In certain embodiments, the liquid formulation further comprises a chelating agent. Exemplary chelating agent include, but are not limited to, ethylenediaminetetraacetic acid (EDTA). However, in certain embodiments, the liquid formulation comprises about 0.02% (w/v) to about 0.4% (w/v) of disodium EDTA.

Exemplary preservatives that can be optionally provided in the liquid formulation include, but are not limited to, methylparaben, propylparaben, or a combination thereof. However, in certain embodiments, the liquid formulation is substantially free of preservatives.

Thus, in certain embodiments, the liquid formulation optionally contains a chelating agent and/or a preservative. However, in certain other embodiments, the liquid formulation is substantially free of chelating agents and/or preservatives.

This disclosure provides a ready-to-administer parenteral liquid formulation comprising morphine sulfate, a tonicity agent, a buffer system, a chelating agent, one or more pH adjusting agents, and water for injection. An exemplary embodiment is provided in Table 1 below.

TABLE 1

| Ingredient | Function | Amount per mL | |
|---|---|---|---|
| Morphine Sulfate, USP | Active Ingredient | 1.0 | mg |
| Sodium Chloride, USP | Tonicity | 9 | mg |
| Disodium EDTA, USP | Chelating agent | 0.2 | mg |
| Citric Acid Monohydrate, USP | Buffering | 0.4 | mg |

| | | |
|---|---|---|
| Sodium Hydroxide, NF | pH adjustment | as needed |
| Hydrochloric Acid, NF | pH adjustment | as needed |
| Water for Injection, USP | Aqueous Vehicle | q.s. ad 1 ml |

### D. Polymeric Infusion Containers

The morphine sulfate formulations disclosed herein are ideally ready-to-administer (RTA) formulations for parenteral administration without the need for reconstitution or further dilution. The ready-to-administer formulations are liquid stored in a pharmaceutically acceptable container, for example, an infusion container such as polymeric infusion container (e.g., intravenous (IV) bag). Diluents can include, for instance, fluids suitable for parenteral administration such as sodium chloride solutions.

Exemplary materials for the containers and container components include, but are not limited to, polyolefins, polysulfone, polycarbonate, polypropylene, polyethylene (such as low density polyethylene (LDPE) or high density polyethylene (HDPE)), polystyrene, and co-polymers thereof. Preferably, the materials for the container and container components are suitable to withstand aseptic filling and not deform during terminal sterilization. In certain embodiments, the polymeric infusion container comprises a polyolefin/styrene block copolymer, such as the polymeric IV bags manufactured by PolyCine OR Sifra.

The infusion container can be a flexible plastic container, optionally with ports and closure system for storing the liquid formulations. Infusion containers can include other conventional components, for example, connection ports, connector caps or connector disks.

The infusion containers may be sealed with any appropriate closure, such as a twist-off stopper. An exemplary twist-off stopper comprises a polypropylene film, such as Inerta® supplied by Technoflex® or equivalent.

In certain embodiments, the morphine sulfate liquid formulation is prepared as a 50 ml fill or a 100 mL fill packaged in a 50 ml or 100 mL, respectively, polymeric infusion container, such as a single port IV bag with a twist-off stopper.

An exemplary polymeric infusion container and closure system for morphine sulfate is provided in Table 2 below.

TABLE 2

| Component | Description |
|---|---|
| IV Bag | Bag: 100 cc, PolyCine APP114/Sifra |
| | multilayer Polyolefin/Styrene |
| | block copolymer, 200 μm |
| Tube | Tube: APP 107, 1000 μm |
| Twist-off stopper | Polypropylene twist-off |

### E. Terminal Sterilization OR Gamma Sterilisation

General procedures for filling the liquid formulation into the polymeric infusion containers and their subsequent processing, for example, terminal sterilization, are known in the art.

Processing techniques of the formulations filled in an infusion container preferably use a sterilization process to destroy or eliminate any microorganisms that may be present in the morphine sulfate formulations following preparation. For example, terminal sterilization can be used to destroy or eliminate any microorganisms that may be present in the sealed polymeric infusion container containing the morphine sulfate liquid formulation. An autoclave is commonly used to accomplish terminal heat-sterilization of drug products in their final packaging.

In certain embodiments, the polymeric infusion container filled with the liquid formulation containing morphine sulfate is terminally sterilized using an autoclave, such as water cascade autoclave OR Air-Steam mixture counter pressure autoclave. The liquid formulations in the polymeric infusion containers can be autoclaved at a temperature ranging from 115° C. to 130° C., preferably at a temperature ranging from 119° C. to 122° C., for a period of time ranging from about 5 to about 30 minutes, alternatively from about 5 to about 15 minutes, alternatively from about 12 to about 15 minutes.

In certain embodiments, a terminal sterilization cycle is based on a D₁₂₁-value or decimal reduction time. In some such embodiments, the terminal sterilization cycle is based on a D₁₂₁-value that is less than 1.5, alternatively less than 1.0, such as about 0.5.

In certain embodiments, terminal sterilization can be characterized by specifying a F₀ value. The F₀ value is the equivalent time in minutes for the specified temperature that gives the same thermal lethality as at 121° C. For example, if a cycle has an F₀ value of 6, the sterilization effectiveness of that cycle is equal to 6 minutes at 121° C. regardless of the process temperature and time used in the cycle. In some such embodiments, the F₀ is less than 20, such as from about 5 to about 15, alternatively from about 6 to about 11. In some such embodiments, the Fois about 6. In some such embodiments, the F₀ is not more than about 15, alternatively not more than about 12, alternatively not more than about 11.

As described herein, it was observed that the morphine sulfate liquid formulations contained in a polymeric infusion container can withstand terminal sterilization cycles up to F₀ of 15 minutes. Thus, in some such embodiments, the F₀ does not exceed 15.

In certain embodiments, the liquid formulation containing morphine sulfate is substantially free of viable microbial contamination after terminal sterilization. Methods for assessing microbial contamination are known in the art and include testing for sterility in accordance with USP <71> using Trypticase Soy Broth (Soybean-Casein Digest Medium), which is suitable for the culture of both fungi and aerobic bacteria and/or Fluid Thioglycolate Medium, which is a medium primarily intended for the culture of anaerobic bacteria.

### F. Compounding Procedure

In one aspect, this disclosure provides a method for manufacturing a terminally sterilized, 1.0 mg/mL presentation of morphine sulfate in 0.9% sodium chloride packaged in a polymeric infusion container such as an RTA IV bag.

In certain embodiments, compounding takes place in a controlled environment in a stainless steel vessel equipped with a mixer.

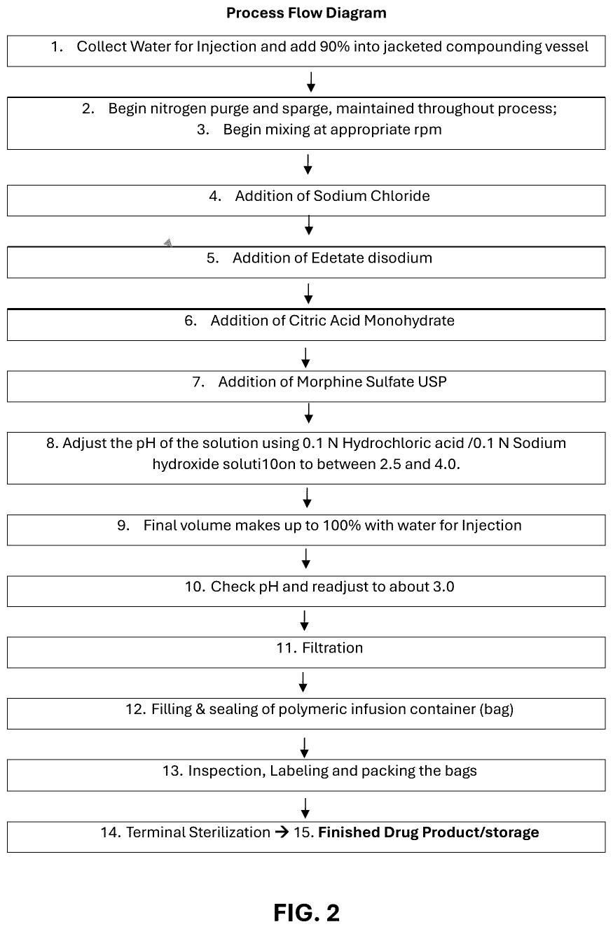

FIG. 2 depicts an exemplary process flow diagram for the manufacture of a terminally sterilized morphine sulfate formulation packaged in an RTA IV bag.

At step 1, add 90% of the water for injection (WFI) to an empty compounding vessel.

At step 2, begin a nitrogen purge and sparge. Maintain both purge and sparge throughout formulation.

At step 3, begin mixing at an appropriate rpm.

At step 4, add calculated amount of sodium chloride to the compounding vessel (e.g., to achieve a final concentration of 9 mg/mL). Mix for at least 5 minutes. Verify dissolution of the sodium chloride.

At step 5, add calculated amount of disodium edetate to the compounding vessel (e.g., to achieve a final concentration of 0.2 mg/mL). Mix for at least 5 minutes. Verify dissolution of the disodium edetate.

At step 6, add calculated amount of Citric Acid Monohydrate to the compounding vessel (e.g., to achieve a final concentration of 0.4 mg/mL). Mix for at least 5 minutes. Verify dissolution of the Citric Acid Monohydrate.

At step 7, add calculated amount of morphine sulfate, USP to the compounding vessel (e.g., to achieve a final concentration of 1.0 mg/ml). Mix for at least 5 minutes. Verify dissolution of the morphine sulfate.

At step 8, measure pH and adjust to around 3.2 (Target around 3.0) using 0.1 N Sodium Hydroxide or 0.1 N hydrochloric acid solution. Mix for at least 5 minutes between each addition.

At step 9, add WFI to QS until final solution weight is reached and mix for at least 10 minutes and confirm the homogeneity of the solution.

At step 10, measure pH and adjust to 2.5-4.0 (Target 3.0) using 0.1 N sodium hydroxide or 0.1 N Hydrochloric Acid, if necessary. Mix for at least 5 minutes between each addition.

Subsequent filtration and filling may occur in a controlled environment.

At step 11, the bulk solution is pressure transferred from the holding vessel through PFA tubing and stainless steel contact parts and 5 inch 0.2 μm cartridge filter housed in stainless steel.

At step 12, the filtered bulk solution is pressurized through the filling setup composed of PFA tubing and stainless steel contact parts, through a stainless steel filling needle and then filled into IV bags. Once filled, the bags will be stoppered with a twist-off stopper.

At step 13, the bags are inspected.

At step 14, filled bags are terminally sterilized using a water cascade autoclave at a temperature of 121° C. and a set F₀ of >6.0.

At step 15, autoclaved bags are dried, inspected, and placed in an aluminum overwrap pouch for storage.

### G. Stability

Morphine sulfate is known to be heat and light sensitive and reactive with strong oxidizing agents. Advantageously, the liquid formulation and containment in a polymeric infusion administration bag, which allows for terminal sterilization of the liquid formulation, provides extended stability and preservation of the liquid morphine sulfate formulation of the invention.

### Degradation Products of Morphine Sulfate.

In at least one aspect, the terminally sterilized liquid formulations disclosed herein are stable during, for example, storage, distribution, and the duration of the product's shelf-life (e.g., up to two years at room temperature/ambient conditions). A stable terminally sterilized liquid formulations may, for example, exhibit less degradation of the active ingredient and/or low amounts of degradation products. Assay and degradation product determination of liquid formulations may be performed using High Performance Liquid Chromatography (“HPLC”) with UV detection

In certain embodiments, the terminally sterilized morphine sulfate liquid formulation packaged in a polymeric infusion container such as an IV bag meets release specifications and/or shelf-life specifications for morphine sulfate, particular related substances, total related substances, and/or individual unknown impurities. In some such embodiments, the amount of morphine sulfate, related substances, and/or individual unknown impurities is determined by HPLC.

Exemplary release specifications for morphine sulfate are at least 93%, at least 94%, at least 95%, at least 96%, at least 97%, at least 98%, or at least 99% of the specified amount of morphine sulfate (e.g., 1.0 mg/mL).

Exemplary release specifications for related compounds, such as any single maximum known or unknown degradation impurities are not more than 0.5%, not more than 0.35%, not more than 0.20%, or not more than 0.15% of the particular related compound.

Exemplary shelf-life specifications for disodium edetate are at least 90%, at least 91%, at least 92%, at least 93%, at least 94%, at least 95%, at least 96%, at least 97%, or at least 98% of the specified amount of disodium edetate (e.g., 0.2 mg/ml).

Exemplary shelf-life specifications for morphine sulfate are at least 90%, at least 91%, at least 92%, at least 93%, at least 94%, at least 95%, at least 96%, at least 97%, or at least 98% of the specified amount of morphine sulfate (e.g., 1.0 mg/mL).

Exemplary shelf-life specifications for sodium chloride are at least 90%, at least 91%, at least 92%, at least 93%, at least 94%, at least 95%, at least 96%, at least 97%, or at least 98% of the specified amount of sodium chloride (e.g., 9.0 mg/ml).

Exemplary shelf-life specifications for total related compounds, which includes all known impurities as well as individual unknown impurities, are not more than 3.0%, not more than 2.0%, not more than 1.5%, not more than 1.25%, not more than 1.0%, not more than 0.75%, or not more than 0.5%.

Stability of a terminally sterilized morphine sulfate liquid formulation packaged in a polymeric infusion container such as an IV bag may be assessed following storage for at least two weeks, at least 1 month, at least 2 months, at least 3 months, at least 6 months, at least 12 months, at least 15 months, at least 18 months, or at least 24 months. In particular, pH, osmolality, morphine concentration, degradation products or other impurities, may be assessed after storage for about 1, 2, 3, 6, 9, 12, 15, 18, 24, 36, and/or 48 months. Storage conditions may be long term, intermediate, or accelerated conditions. In particular, storage conditions may be, for example, 25° C.±2° C. and 40% relative humidity (RH)±5% RH, 30° C.±2° C. and 65% RH±5% RH, and/or 40° C.±2° C. and not more than 25% RH.

In certain embodiments, storage of the terminally sterilized morphine sulfate liquid formulation packaged in a polymeric infusion container is at 25° C.±2° C. and 40%±5% relative humidity for between about 1 month and about 48 months, between about 2 months and about 36 months, or between about 3 months and about 24 months. In certain embodiments, storage of the terminally sterilized morphine sulfate liquid formulation packaged in a polymeric infusion container is at 25° C.±2° C. and 40%±5% relative humidity for at least 3 months, at least 6 months, at least 12 months, at least 15 months, at least 18 months, or at least 24 months. In some such embodiments, storage stability is assessed at about 3, 6, 9, 12, 15, 18, 24, and/or 36 months.

In certain embodiments, storage of the terminally sterilized morphine sulfate liquid formulation packaged in a polymeric infusion container is at 30° C.±2° C. and 65%±5% relative humidity for between about 1 month and about 24 months or between about 1 month and about 12 months. In certain embodiments, storage of the terminally sterilized morphine sulfate liquid formulation packaged in a polymeric infusion container is at 30° C.±2° C. and 65%±5% relative humidity for at least 2 months, at least 3 months, at least 6 months, or at least 12 months. In some such embodiments, storage stability is assessed at about 1, 2, 3, 6, and/or 12 months.

In certain embodiments, storage of the terminally sterilized morphine sulfate liquid formulation packaged in a polymeric infusion container is at 40° C.±2° C. and not more than 25% relative humidity for between about 1 month and about 12 months or between about 1 month and about 6 months. In certain embodiments, storage of the terminally sterilized morphine sulfate liquid formulation packaged in a polymeric infusion container is at 40° C.±2° C. and not more than 25% relative humidity for at least 2 months, at least 3 months, or at least 6 months. In some such embodiments, storage stability is assessed at about 1, 2, 3, and/or 6 months.

Storage stability assessments may include pH, osmolality, morphine concentration, degradation products or other impurities.

In certain embodiments, the terminally sterilized morphine sulfate liquid formulation packaged in a polymeric infusion container such as an IV bag has a pH in the range of about 2.5 to about 6.5, alternatively, from about 2.5-4.0, or alternatively from about 3.0 to about 4.0 after storage for up to 6 months, up to 9 months, up to 12 months, up to 15 months, up to 18 months, or up to 24 months under long-term stability conditions. In some such embodiments, the terminally sterilized morphine sulfate liquid formulation packaged in a polymeric infusion container such as an IV bag has a pH of about 3.0, about 3.2, about 3.3, after storage for up to 6 months, up to 9 months, up to 12 months, up to 15 months, up to 18 months, or up to 24 months under long-term stability conditions.

In certain embodiments, the pH of the terminally sterilized morphine sulfate liquid formulation packaged in a polymeric infusion container such as an IV bag does not materially change over time under long-term stability conditions.

In certain embodiments, the terminally sterilized morphine sulfate liquid formulation packaged in a polymeric infusion container such as an IV bag retains at least 90%, alternatively at least 91%, alternatively at least 92%, alternatively at least 93%, alternatively at least 94%, alternatively at least 95%, alternatively at least 96%, alternatively at least 97%, or alternatively at least 98% of the morphine sulfate in the liquid formulation after storage for up to 6 months, up to 9 months, up to 12 months, up to 15 months, up to 18 months, or up to 24 months under long-term stability conditions as determined by HPLC.

In some such embodiments, the amount of morphine sulfate retained following storage is with reference to the morphine sulfate concentration prior to storage. In some such embodiments, the morphine sulfate concentration prior to storage is from about 0.01 mg/ml to about 2.0 mg/ml, alternatively from about 0.05 mg/ml to about 1.0 mg/mL, or alternatively from about 0.1 mg/ml to about 1.0 mg/mL. In some such embodiments, the morphine sulfate concentration prior to storage is 1.0 mg/mL.

In certain embodiments, the amount of morphine sulfate in the terminally sterilized morphine sulfate liquid formulation packaged in a polymeric infusion container such as an IV bag does not materially change over time under long-term stability conditions.

In certain embodiments, the terminally sterilized morphine sulfate liquid formulation packaged in a polymeric infusion container such as an IV bag contains not more than not more than 1.0%, not more than 0.9%, not more than 0.8%, not more than 0.7%, not more than 0.6%, not more than 0.5%, not more than 0.4%, not more than 0.3%, not more than 0.2%, or not more than 0.1% of any individual related substance after storage for up to 6 months, up to 9 months, up to 12 months, up to 15 months, up to 18 months, or up to 24 months under long-term stability conditions as determined by HPLC.

In certain embodiments, the liquid formulation is stable for at least 12 months or at least 18 months when stored at 25° C.±2° C. and 40% relative humidity (RH)±5% RH. In some such embodiments, the liquid formulation is stable for up to 24 months when stored at 25° C.±2° C. and 40% relative humidity (RH)±5% RH.

In some such embodiments, the liquid formulation retains at least 90% w/w, preferably at least 95% w/w, and more preferably at least 98% of the morphine sulfate and has not more than 10% w/w, preferably not more than 5% w/w, and more preferably not more than 2% w/w of total related substances after storage at 25° C.±2° C. and 40% RH±5% RH for 12, 18, or 24 months.

In some such embodiments, the liquid formulation retains at least 90% w/w, preferably at least 95% w/w, and more preferably at least 98% of the morphine sulfate and has not more than 0.8% w/w, preferably not more than 0.5% w/w, and more preferably not more than 0.3% w/w of any individual related substance after storage at 25° C.±2° C. and 40% RH±5% RH for 12, 18, or 24 months.

In some such embodiments, the pH of the liquid formulation after storage at 25° C.±2° C. and 40% RH±5% RH for 12, 18, or 24 months is in the range of about 2.5 to about 6.5, alternatively, from about 2.5-4.0, or alternatively from about 3.0 to about 4.0. In an exemplary embodiment, the pH of the liquid formulation after storage at 25° C.±2° C. and 40% RH±5% RH for 12, 18, or 24 months is in the range of about 3.0 to about 4.0, preferably about 3.0 to 3.4.

### H. Methods of Use

In at least one aspect, this disclosure provides methods for the management of pain using the liquid formulations described herein. Liquid formulations comprising morphine or a pharmaceutically acceptable salt thereof may be used for the management of pain and, in particular, for the management of pain severe enough to require an opioid analgesic and for which alternate treatments are inadequate. Liquid formulations described herein may preferably be parenterally administered to a subject in need thereof. In particular, liquid formulations described herein may be intravenously administered to a subject in need thereof.

Thus, in certain embodiments, the methods comprise parenterally administering to a subject in need of management of pain the terminally sterilized morphine sulfate liquid formulations described herein. In some such embodiments, the methods comprise intravenously administering to a subject in need of management of pain the terminally sterilized morphine sulfate liquid formulations described herein.

In certain embodiments, the subject is a human.

The liquid formulations, methods, and uses described herein will be better understood by reference to the following exemplary embodiments and examples, which are included as an illustration of and not a limitation upon the scope of the invention.

### EXEMPLARY EMBODIMENTS

Exemplary Embodiment 1. A ready-to-administer parenteral liquid formulation comprising (a) morphine sulfate; (b) a tonicity adjusting agent; (c) a chelating agent; and (d) a buffer system; wherein the liquid formulation has a pH of 2.5-4.0, wherein the liquid formulation has been terminally sterilized in a polymeric infusion container, and wherein the liquid formulation is stable for at least 6 months when stored at 25° C.±2° C. and 40% relative humidity (RH)±5% RH.
Exemplary Embodiment 2. The liquid formulation of embodiment 1, wherein the liquid formulation has a pH of 2.5-4.0, preferably 3.0-3.2.
Exemplary Embodiment 3. The liquid formulation of embodiment 1 or embodiment 2, wherein the buffer system comprises a phosphate buffer, a citrate buffer, an acetate buffer, a histidine buffer, or a combination thereof.
Exemplary Embodiment 4. The liquid formulation of embodiment 1 or embodiment 2, wherein the buffer system comprises citric acid, sodium citrate, acetic acid, sodium acetate, or a combination thereof.
Exemplary Embodiment 5. The liquid formulation of embodiment 1 or embodiment 2, wherein the buffer system is a citrate buffer.
Exemplary Embodiment 6. The liquid formulation of embodiment 5, wherein the liquid formulation comprises from about 0.1 mg/ml to about 1.0 mg/ml of the citrate buffer.
Exemplary Embodiment 7. The liquid formulation of embodiment 1 or embodiment 2, wherein the liquid formulation comprises about 0.04% (w/v) of citric acid monohydrate.
Exemplary Embodiment 8. The liquid formulation of any one of embodiments 1-7, wherein the tonicity adjusting agent comprises sodium chloride, calcium chloride, magnesium chloride, or a combination thereof.
Exemplary Embodiment 9. The liquid formulation of any one of embodiments 1-7, wherein the tonicity adjusting agent comprises sodium chloride.
Exemplary Embodiment 10. The liquid formulation of embodiment 9, wherein the liquid formulation comprises about 9 mg/ml sodium chloride.
Exemplary Embodiment 11. The liquid formulation of any one of embodiments 1-10, wherein the liquid formulation has an osmolality of from about 275 mOsmol/kg to about 310 mOsmol/kg.
Exemplary Embodiment 12. The liquid formulation of any one of embodiments 1-11, wherein the liquid formulation comprises from about 0.05 mg/ml to about 1.0 mg/ml of morphine sulfate.
Exemplary Embodiment 13. The liquid formulation of embodiment 12, wherein the liquid formulation comprises 1.0 mg/mL of morphine sulfate.
Exemplary Embodiment 14. A ready-to-administer parenteral liquid formulation comprising: (a) 0.2 mg/ml of morphine sulfate; (b) about 9 mg/ml of sodium chloride; (c) 0.20 mg/ml of disodium edetate; and (d) 0.40 mg/ml of citric acid monohydrate; wherein the liquid formulation has a pH of 2.5 to 4.0, wherein the liquid formulation has been terminally sterilized in a polymeric infusion container, and wherein the liquid formulation is stable for at least 6 months when stored at 25° C.±2° C. and not more than 40% RH.
Exemplary Embodiment 15. The liquid formulation of embodiment 14, wherein the liquid formulation has a pH of 2.5 to 4.0
Exemplary Embodiment 16. The liquid formulation of any one of embodiments 1-15, further comprising a pH adjusting agent.
Exemplary Embodiment 17. The liquid formulation of any one of embodiments 1-16, wherein the liquid formulation retains at least 95% w/w, preferably at least 98% w/w, of the morphine sulfate and has not more than 5% w/w, preferably not more than 2% w/w, of total related substances after storage at 25° C.±2° C. and not more than 40% RH for at least 6 months, preferably at least 12 months.
Exemplary Embodiment 18. A method of treating a patient in need of management of pain comprising parenterally administering to the patient the liquid formulation of any one of embodiments 1-17.
Exemplary Embodiment 19. The method of embodiment 18, wherein the pain is moderate to severe pain due to surgery, cancer, trauma (soft tissue and bone), biliary colic, myocardial infarction, burns, or renal colic.
Exemplary Embodiment 20. A polymeric infusion container filled with the liquid formulation of any one of embodiments 1-17.
Exemplary Embodiment 21. A method for manufacturing a ready-to-administer parenteral liquid formulation containing morphine sulfate comprising: providing the liquid formulation containing morphine sulfate in a polymeric infusion container, wherein the liquid formulation has an initial pH of 3.0 to 4.0; and terminally sterilizing the liquid formulation containing morphine sulfate in the polymeric infusion container.
Exemplary Embodiment 22. The method of embodiment 21, wherein the liquid formulation has an initial pH of 2.5-4.0, preferably 3.0 to 3.3.
Exemplary Embodiment 23. The method of embodiment 21 or embodiment 22, wherein the liquid formulation further comprises a buffer system.
Exemplary Embodiment 24. The method of embodiment 23, wherein the buffer system is a citrate buffer.
Exemplary Embodiment 25. The method of embodiment 24, wherein the liquid formulation comprises from about 0.1 mg/ml to about 1.0 mg/ml of the citrate buffer.
Exemplary Embodiment 26. The method of embodiment 21, wherein the liquid formulation comprises about 0.02% (w/v) of Disodium edetate and about 0.04% (w/v) of citric acid monohydrate.
Exemplary Embodiment 27. The method of any one of embodiments 21-26, wherein the liquid formulation further comprises a tonicity adjusting agent.
Exemplary Embodiment 28. The method of embodiment 27, wherein the tonicity adjusting agent comprises sodium chloride.
Exemplary Embodiment 29. The method of any one of embodiments 21-26, wherein the liquid formulation comprises about 9 mg/ml sodium chloride.

### EXAMPLES

In order to demonstrate the practice of the subject matter disclosed herein, the following examples have been prepared and tested. The examples should not, however, be viewed as limiting the scope of the invention.

### Example 1: Formulation for Morphine Sulfate Injection

This study characterized the impact of terminal sterilization on a 1.0 mg/ml of morphine sulfate. The drug product was prepared and analyzed to investigate the suitability of the formulation and container closure system.

The batch was prepared using the formulation in Table 3 according to the procedure described herein. Briefly, the process was performed under yellow lighting and involved the following steps: sparged water with nitrogen for 20 minutes; added 90% final volume of water to the vessel; added sodium chloride and mixed to dissolve; added morphine sulfate (as a concentrate in water) and mixed to dissolve; checked pH and adjusted to between 2.5 to 6.5 with hydrochloric acid; brought solution to q.s. with water; sparged solution with nitrogen for five minutes; and stored bulk solution under a nitrogen headspace until filling into bags.

TABLE 3
Formulation for Morphine Sulfate
in 0.9% Sodium Chloride Injection

| Ingredient | Function | Amount per mL | |
|---|---|---|---|
| Morphine Sulfate, USP | Active Ingredient | 1.0 | mg |
| Sodium Chloride, USP | Tonicity | 9 | mg |

| | | |
|---|---|---|
| Sodium Hydroxide, NF | pH adjustment | as needed |
| Hydrochloric Acid, NF | pH adjustment | as needed |
| Water for Injection, USP | Aqueous Vehicle | q.s. ad 1 mL |

The bulk solution was filled into single port IV bags and closed with a polypropylene twist-off.

The results of the analysis are presented in Table 4 and Table 5.

TABLE 4
Stability study at Accelerated stability condition (40° C./NMT 25% RH)

| Test | Specifications | Initial | 2 Month | 3 Month | 6 Month |
|---|---|---|---|---|---|
| Description | Clear, colorless to slightly | CCS | CCS | CCS | CCS |
| | yellow solution visually | | | | |
| | free from particulates | | | | |
| pH of Solution | 2.5-6.5 | 5.28 | 5.20 | 5.12 | 5.05 |
| Osmolality (mOsmol/Kg) | 250-350 | NA | 289 | 290 | 291 |
| Visual Inspection | Should be free of | Free of | Free of | Free of | Free of |
| (Sub-Visible Particles) | visible particles | visible | visible | visible | visible |
| | | particles | particles | particles | particles |

Particulate Matter (USP 43 <788> Test 1.B)

| | | | | | |
|---|---|---|---|---|---|
| ≥10 μm size particles | NMT 6000 particles | 53.33 | 20.00 | 66.67 | 53.33 |
| | per container | | | | |
| ≥25 μm size particles | NMT 600 particles | 6.67 | 6.67 | 13.33 | 13.33 |
| | per container | | | | |
| % Assay of Morphine | 90.0-110.0% | 101.5 | 101.6 | 99.6 | 97.8 |
| Sulfate | | | | | |
| % Assay of Sodium | 90.0-110.0% | 100.6 | 100.0 | 100.0 | 100.0 |
| Chloride | | | | | |

Organic Impurities by HPLC

| | | | | | |
|---|---|---|---|---|---|
| Any unspecified | NMT 0.2% | 0.03 | 0.16 | 0.07 | 0.16 |
| degradation product | | | | | |
| Total degradation | NMT 1.8% | 0.2 | 0.4 | 0.3 | 0.3 |
| products | | | | | |

TABLE 5
Stability study at Long term stability condition (25° C./40% RH)

| Test | Specifications | Initial | 2 Month | 3 Month | 6 Month |
|---|---|---|---|---|---|
| Description | Clear, colorless to slightly | CCS | CCS | CCS | CCS |
| | yellow solution visually | | | | |
| | free from particulates | | | | |
| pH of Solution | 2.5-6.5 | 5.28 | 5.31 | 5.41 | 5.17 |
| Osmolality (mOsmol/Kg) | 250-350 | NA | 288 | 289 | 289 |
| Visual Inspection | Should be free of | Free of | Free of | Free of | Free of |
| (Sub-Visible Particles) | visible particles | visible | visible | visible | visible |
| | | particles | particles | particles | particles |

Particulate Matter (USP 43 <788> Test 1.B)

| | | | | | |
|---|---|---|---|---|---|
| ≥10 μm size particles | NMT 6000 particles | 53.33 | 60.00 | 100.00 | 46.67 |
| | per container | | | | |
| ≥25 μm size particles | NMT 600 particles | 6.67 | 0.00 | 13.33 | 13.33 |
| | per container | | | | |
| % Assay of Morphine | 90.0-110.0% | 101.5 | 101.4 | 99.8 | 97.9 |
| Sulfate | | | | | |
| % Assay of Sodium | 90.0-110.0% | 100.6 | 100.6 | 100.0 | 100.6 |
| Chloride | | | | | |

Organic Impurities by HPLC

| | | | | | |
|---|---|---|---|---|---|
| Any unspecified | NMT 0.2% | 0.03 | 0.05 | 0.04 | 0.07 |
| degradation product | | | | | |
| Total degradation | NMT 1.8% | 0.2 | 0.3 | 0.3 | 0.3 |
| products | | | | | |

There was no significant change in pH between the initial and 6 months stored samples. There were no significant differences in appearance and assay for bulk solution stored in the IV bag.

Based on literature information morphine degrades in aqueous solutions with the formation of mainly pseudomorphine, to a lesser extent morphine-N-oxide and probably apomorphine. From the study of the kinetics of morphine degradation it was concluded that the degradation of morphine is accelerated in the presence of oxygen and at higher pH of the solution, whereas temperature and light have only a minor influence on the degradation rate.” (Stability and compatibility of morphine; A Vermeire, J P Remon; Int J Pharm, 1999 Sep. 30; 187 (1): 17-51) Hence, plan to take further development trials (Example 2) of morphine sulfate in 0.9% Sodium Chloride Injection, 1 mg/ml with buffer and chelating agent are planned to better control pH and impurity profile.

### Example 2: Bulk Solution

This study characterized the impact of terminal sterilization on a 1.0 mg/ml bulk solution of morphine sulfate with buffer and chelating agent. The drug product was prepared and analyzed to investigate the suitability of the formulation and container closure system.

The batch was prepared using the formulation in Table 6 according to the procedure described herein. Briefly, the process was performed under yellow lighting and involved the following steps: sparged water with nitrogen; added 90% final volume of water to the vessel; added Sodium Chloride and mixed to dissolve; added disodium edetate and mixed to dissolve; added citric acid monohydrate and mixed to dissolve; added morphine sulfate (as a concentrate in water) and mixed to dissolve; checked pH and adjusted to about 3.0; brought solution to q.s. with water; sparged solution with nitrogen for five minutes; and stored bulk solution under a nitrogen headspace until filling into bags.

TABLE 6
Formulation for Morphine Sulfate
in 0.9% Sodium Chloride Injection

| Ingredient | Function | Amount per mL | |
|---|---|---|---|
| Morphine Sulfate, USP | Active Ingredient | 1.0 | mg |
| Sodium Chloride, USP | Tonicity | 9 | mg |
| Disodium Edetate USP | Chelating agent | 0.2 | mg |
| Citric Acid Monohydrate, USP | Buffering | 0.40 | mg |

| | | |
|---|---|---|
| Sodium Hydroxide, NF | pH adjustment | pH adjustment |
| Hydrochloric Acid, NF | pH adjustment | pH adjustment |
| Water for Injection, USP | Aqueous Vehicle | q.s. ad 1 ml |

The bulk solution was filled into single port IV PP bags and closed with a polypropylene twist-off.

The finished bags were then terminally sterilized in a autoclave using an air overpressure cycle with a target F₀ of 15 minutes. The finished bags were then analyzed.

A control sample not exposed to terminal sterilization stored in an IV bag was also tested. The results of the analysis are presented in Table 7.

TABLE 7
Analytical results for Aseptic vs Terminally sterilized
vs Two time terminally sterilized product.

| | | | Two time |
|---|---|---|---|
| | | Terminally | terminally |
| Test | Aseptic | sterilized | sterilized |
| Description | CCS | CCS | CCS |
| pH of Solution | 3.03 | 3.03 | 3.01 |
| Osmolality (mOsmol/Kg) | 292 | 291 | 290 |
| Visual Inspection | Free of visible | Free of visible | Free of visible |
| (Sub-Visible Particles) | particles | particles | particles |
| ≥10 μm size particles | 273.33 | 10.00 | 103.33 |
| ≥25 μm size particles | 13.33 | 0.00 | 3.33 |
| % Assay of Morphine | 102.5 | 101.9 | 101.9 |
| Sulfate | | | |
| % Assay of Sodium | 101.3 | 100.0 | 100.0 |
| Chloride | | | |
| Morphine N-Oxide | ND | ND | ND |
| Morphinone | ND | ND | ND |
| Pseudomorphine | 0.01 | 0.02 | 0.02 |
| Codeinone | ND | ND | ND |
| Any unspecified | 0.09 | 0.10 | 0.10 |
| degradation product | | | |
| Total degradation | 0.2 | 0.2 | 0.2 |
| products | | | |

CCS: Clear Colorless Solution,
ND = Not Detected

Currently available formulations contain a 0.8% citrate buffer system. These results confirm that a 2 times reduction of buffer strength had no impact on the analytical profile after terminal sterilization.

There was no significant change in pH after terminal sterilization in an IV bag.

There were no significant differences in appearance and assay for bulk solution terminally sterilized in glass compared to the same performed in the IV bag. The related substances were observed to be slightly higher for bulk solution terminally sterilized in the IV bag indicating that the IV bag may be more suitable for terminal sterilization of this formulation.

The results indicate that the disclosed formulations can withstand terminal sterilization cycles up to F₀ of 15 minutes. Longer cycles will result in higher levels of related substances, and therefore the terminal sterilization cycle time should be minimized.

### Example 3: Stability Study

The effect of terminal sterilization on the stability of the product was characterized. Samples were prepared in accordance with Example 2 and filled into IV bags. Bags were subsequently stored horizontally at 25° C. and 40% RH and at 40° C. and NMT 25% RH.

Samples were assessed by morphine sulfate assay at 3, 6, 9, 12, 15, 18, and 24 months. pH and individual related substances were also assessed. Tables 8-9 show stability over time for each lot tested.

TABLE 8
Stability study at Accelerated stability condition (40° C./NMT 25% RH)

| Test | Specifications | Initial | 1 Month | 3 Month |
|---|---|---|---|---|
| Description | Clear, colorless to slightly | CCS | CCS | CCS |
| | yellow solution visually | | | |
| | free from particulates | | | |
| pH of Solution | 2.5-6.5 | 3.03 | 3.02 | 3.12 |
| Osmolality (mOsmol/Kg) | 250-350 | 292 | 292 | 292 |
| ≥10 μm size particles | NMT 6000 particles | 273.33 | 26.67 | 140.00 |
| | per container | | | |
| ≥25 μm size particles | NMT 600 particles | 13.33 | 6.67 | 20.00 |
| | per container | | | |
| % Assay of Morphine | 90.0-110.0% | 102.5 | 103.3 | 98.5 |
| Sulfate | | | | |
| % Assay of EDTA | 90.0-110.0% | 100.2 | 101.1 | 99.9 |
| % Assay of Sodium | 90.0-110.0% | 101.3 | 100.0 | 100.0 |
| Chloride | | | | |
| Morphine N-Oxide | NMT 0.2% | ND | ND | ND |
| Morphinone Sulfate | NMT 0.2% | ND | ND | ND |
| Pseudomorphine Sulfate | NMT 0.2% | 0.01 | 0.01 | 0.04 |
| Codeinone | NMT 0.2% | ND | ND | ND |
| Codeine Sulfate | NMT 0.2% | 0.09 | 0.10 | 0.10 |
| Any unspecified | NMT 0.2% | 0.02 | 0.02 | 0.02 |
| degradation product | | | | |
| Total degradation | NMT 1.8% | 0.2 | 0.2 | 0.2 |
| products | | | | |

TABLE 9
Stability study at Long term stability condition (25° C./40% RH)

| Test | Specifications | Initial | 1 Month | 3 Month |
|---|---|---|---|---|
| Description | Clear, colorless to slightly | CCS | CCS | CCS |
| | yellow solution visually | | | |
| | free from particulates | | | |
| pH of Solution | 2.5-6.5 | 3.03 | 3.02 | 3.15 |
| Osmolality (mOsmol/Kg) | 250-350 | 292 | 291 | 290 |
| ≥10 μm size particles | NMT 6000 particles | 273.33 | 13.33 | 80.00 |
| | per container | | | |
| ≥25 μm size particles | NMT 600 particles | 13.33 | 0.00 | 0.00 |
| | per container | | | |
| % Assay of Morphine | 90.0-110.0% | 102.5 | 103.1 | 98.8 |
| Sulfate | | | | |
| % Assay of EDTA | 90.0-110.0% | 100.2 | 101.1 | 100.9 |
| % Assay of Sodium | 90.0-110.0% | 101.3 | 100.6 | 100.0 |
| Chloride | | | | |
| Morphine N-Oxide | NMT 0.2% | ND | ND | ND |
| Morphinone Sulfate | NMT 0.2% | ND | ND | ND |
| Pseudomorphine Sulfate | NMT 0.2% | 0.01 | 0.02 | 0.03 |
| Codeinone | NMT 0.2% | ND | ND | ND |
| Codeine Sulfate | NMT 0.2% | 0.09 | 0.10 | 0.09 |
| Any unspecified | NMT 0.2% | 0.02 | 0.02 | 0.018 |
| degradation product | | | | |
| Total degradation | NMT 1.8% | 0.2 | 0.2 | 0.2 |
| products | | | | |

The above disclosure and example generally describe the present invention and is provided for purposes of illustration and is not intended to limit the scope of the invention. The invention described herein may be practiced in the absence of any element or elements, limitation or limitations which is not specifically disclosed herein. Thus, for example, in each instance herein, any of the terms “comprising,” “consisting essentially of,” and “consisting of” may be replaced with either of the other two terms. The terms and expressions are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the claims.

1. A ready-to-administer parenteral liquid formulation comprising:
(a) 0.5 to 1.5 mg/ml of morphine sulfate;
(b) a chelating agent;
(c) a tonicity adjusting agent; and
(d) a buffer system;
wherein the liquid formulation has a pH of 3.0 to 3.2
wherein the liquid formulation has been terminally sterilized in a polymeric infusion container,
wherein the liquid formulation is stable for at least 6 months when stored at 25° C.±2° C. and 40% relative humidity (RH)±5% RH,
wherein the liquid formulation comprises about 0.02% w/v of disodium edetate and about 0.04% w/v of citric acid monohydrate, and
wherein the ready-to-administer parenteral liquid formulation is indicated for continuous intravenous infusion.

2-4. (canceled)

5. The liquid formulation of claim 1, wherein the tonicity adjusting agent comprises sodium chloride.

6. The liquid formulation of claim 5, wherein the liquid formulation comprises about 9 mg/mL sodium chloride.

7. The liquid formulation of claim 1, wherein the liquid formulation has an osmolality of from about 275 mOsmol/kg to about 310 mOsmol/kg.

8. (canceled)

9. The liquid formulation of claim 1, wherein the liquid formulation comprises 1.0 mg/mL of morphine sulfate.

10. The liquid formulation of claim 1, wherein the liquid formulation retains at least 95% w/w of the morphine sulfate and has not more than 5% w/w of total related substances after storage at 25° C.±2° C. and not more than 40% RH for at least 6 months.

11. The liquid formulation of claim 1, wherein the liquid formulation retains at least 98% w/w of the morphine sulfate and has not more than 2% w/w of total related substances after storage at 25° C.±2° C. and not more than 40% RH for at least 6 months.

12. A ready-to-administer parenteral liquid formulation comprising:
(a) 1.0 mg/ml of morphine sulfate;
(b) 9 mg/mL of sodium chloride;
(c) 0.20 mg/mL of Disodium Edetate; and
(d) 0.40 mg/mL of citric acid monohydrate;
wherein the liquid formulation has a pH of 3.0 to 3.2,
wherein the liquid formulation has been terminally sterilized in a polymeric infusion container,
wherein the liquid formulation is stable for at least 6 months when stored at 25° C.±2° C. and not more than 40% RH; and
wherein the ready-to-administer parenteral liquid formulation is indicated for continuous intravenous infusion.

13. (canceled)

14. The liquid formulation of claim 12, further comprising a pH adjusting agent.

15. The liquid formulation of claim 12, wherein the liquid formulation retains at least 95% w/w of the morphine sulfate and has not more than 5% w/w of total related substances after storage at 25° C.±2° C. and not more than 40% RH for at least 6 months.

16. The liquid formulation of claim 12, wherein the liquid formulation retains at least 98% w/w of the morphine sulfate and has not more than 2% w/w of total related substances after storage at 25° C.±2° C. and not more than 40% RH for at least 6 months.

17. A method of treating a patient in need of management of pain comprising parenterally administering to the patient the liquid formulation of claim 1.

18. The method of claim 17, wherein the pain is moderate to severe pain due to surgery, cancer, trauma or burns.

19. A method of treating a patient in need of management of pain comprising parenterally administering to the patient the liquid formulation of claim 12.

20. A polymeric infusion container filled with the liquid formulation of claim 1.
